(19) United States
(12) Patent Application Publication
(10) Pub. No.: US 2013/0209986 A1
(43) Pub. Date: Aug. 15, 2013

(54) METHOD FOR ENHANCING BUTYRATE PRODUCTION BY CLOSTRIDIUM TYROBUTYRICUM

(71) Applicant: KOREA INSTITUTE OF SCIENCE AND TECHNOLOGY, (US)

(72) Inventors: Young Soon UM, Seoul (KR); Byoung In SANG, Seoul (KR); Yun Je KIM, Seoul (KR); Ok Kyoung CHOI, Seoul (KR)

(73) Assignee: KOREA INSTITUTE OF SCIENCE AND TECHNOLOGY, Seoul (KR)

(21) Appl. No.: 13/749,867

(22) Filed: Jan. 25, 2013

(30) Foreign Application Priority Data
Feb. 15, 2012 (KR) 10-2012-0015420

(51) Int. Cl. C12P 7/52

(52) U.S. Cl. CPC C12P 7/52
USPC 435/3; 435/141

(57) ABSTRACT

Disclosed is a method for enhancing a butyrate production by Clostridium tyrobutyricum strains, comprising adding an electron transfer mediator on a medium of Clostridium tyrobutyricum strains and fermenting the same; or providing electrons to the Clostridium tyrobutyricum strains with the aid of a reduction electrode (cathode) and fermenting the same for thereby enhancing the production of butyrate, by way of which it is possible to enhance the production of clostridium tyrobutyricum.

### CROSS-REFERENCE TO RELATED APPLICATION

This application claims priority to Korean Patent Application No. 10-2012-0015420, filed on Feb. 15, 2012, and all the benefits accruing therefrom under 35 U.S.C. §119, the contents of which in its entirety are herein incorporated by reference.

### BACKGROUND

1. Field

The present invention relates to a novel method for enhancing the production of butyrate.

2. Description of the Related Art

The environmentally friendly biological fuel production by way of the fermentation of microorganism becomes a big issue in its applications in order to effectively cope with the lacking of energy. Most of fermentation byproducts usually used as a biological fuel is a reduced substance produced by receiving electrons from a substrate. In order to enhance the production of a targeted fermentation substance, it is necessary to enable the flow of the electrons from a substrate to a production line of reduced substances in the metabolic process of microorganism to be a flow of a desired fermentation byproduct.

It is reported that the production of 1, 3-propanediol using Clostridium butyricum strain can be enhanced by using electron transfer mediators (Reimann A, Biebl H, Deckwer W. 1996. Influence of iron, phosphate and methyl viologen on glycerol fermentation of Clostridium butyricum. Appl. Microbiol. Biotechnol. 45(1):47-50). There is another report showing that Methyl Viologen (MV) can help enhance the production of butanol by using Clostridium acetobutylicum (Tashiro Y, Shinto H, Hayashi M, Baba S-i, Kobayashi G, Sonomoto K. 2007. Novel high-efficient butanol production from butyrate by non-growing Clostridium saccharoperbutylacetonicum N1-4 (ATCC 13564) with methyl viologen. J. Biosci. Bioeng. 104(3):238-240).

In recent years, diverse researches are being conducted on generating a reducing powder in such a way to supply electrons from a reduction electrode to microorganisms (Lovley DR. 2011. Powering microbes with electricity: direct electron transfer from electrodes to microbes. Environ. Microb. Rep. 3(1):27-35.). Diverse attempts so as to supply a reducing power by providing electrons to microorganisms with the aid of a reduction electrode are being conducted in a way that hexa-valent uranium is transformed to tetra-valent uranium thus processing the same in a no-flow state and in the ways of a denitrification and a dehydrohalogenation, and their applicable ranges are being expanded.

No methods for enhancing the production of butyrate by supplying a reducing power to Clostridium tyrobutyricum are yet disclosed.

### SUMMARY

It is an object of the present invention to provide a method for enhancing the production of butyrate with microorganisms.

It is another object of the present invention to provide a method for enhancing the production of butyrate with Clostridium tyrobutyricum strains.

It is further another object of the present invention to provide a method for enhancing the production of butyrate in such a way to provide a reducing power to Clostridium tyrobutyricum strains.

To achieve the above objects, there is provided a method for enhancing a butyrate production by Clostridium tyrobutyricum strains which comprises adding an electron transfer mediator on a medium of Clostridium tyrobutyricum strains and fermenting the same; or providing electrons to the Clostridium tyrobutyricum strains with the aid of a reduction electrode (cathode) and fermenting the same for thereby enhancing the production of butyrate.

The technology of the present invention is advantageous to enhance the production of butyrate with Clostridium tyrobutyricum.

### BRIEF DESCRIPTION OF THE DRAWINGS

The above and other objects, features and advantages of the present disclosure will become apparent from the following description of certain exemplary embodiments given in conjunction with the accompanying drawings, in which:

FIG. 1 shows a dendrogram of Clostridium tyrobutyricum;

FIG. 2 shows a view illustrating the production amount of butyrate and biological gas in case of the fermentation by adding electron transfer mediators to a Clostridium tyrobutyricum culture medium (MV; methyl viologen, NR; neutral red, AQ; Anthraquinone);

FIG. 3 shows a view illustrating a schematic procedure in which Clostridium tyrobutyricum helps enhance the production of butyrate from sucrose in a reduction electrode system;

FIG. 4 shows a graph illustrating a result after Clostridium tyrobutyricum has helped increase the production of butyrate in a reduction electrode system using a neutral red;

FIG. 5 shows a photo illustrating NR_{red} after it is reduced by a reducing electrode and turns yellow and NRₒₓ after it is oxidized and turns red as electrons are discharged in microorganisms.

### DETAILED DESCRIPTION OF EMBODIMENTS

Hereinafter, the exemplary embodiments of the present disclosure will be described in detail with reference to the accompanying drawings.

The method for enhancing the production of butyrate using Clostridium tyrobutyricum according to the present invention comprises adding an electron transfer mediator on a medium of Clostridium tyrobutyricum strains and fermenting the same; and providing electrons to the Clostridium tyrobutyricum strains with the aid of a reduction electrode (cathode) and fermenting the same for thereby enhancing the production of butyrate.

The method for enhancing the production of butyrate according to the present invention is directed to enhancing the production of butyrate, but it might be a method which helps produce a smaller amount of byproducts or the byproducts cannot be produced. A liquid byproduct might be acetic acid. Gaseous byproducts might be hydrogen and carbon dioxide. If the byproducts, in particular the liquid byproducts, are less produced or are not produced, it is advantageous in that the separation and purification of butyrate become easier, and the yield of butyrate can be increased. Since the byproducts of acetic acid, etc. are significantly or are not produced at all while the production of butyrate can be enhanced, it can be applied for the sake of various applications. For example, the present invention can be well applied to an application which is intended to avoid the production of byproducts such as acetic acid, etc.

The electron transfer mediators might be Methyl Viologen (MV).

The electron transfer mediators can be selected depending on what microorganism strain is used or what substance is produced. An electron transfer mediator which is proper to enhance the production of butyrate with Clostridium tyrobutyricum is Methyl Viologen (MV). It is possible to significantly enhance the production of butyrate using Clostridium tyrobutyricum in such a way to simply add MV to the culture medium. In contrast, it is impossible to enhance the production of butyrate with Clostridium tyrobutyricum in such a way to add NR or Anthraquinone-1, 5-disulfonic acid (AQDS).

The concentration of MV might be above 0.01 mM, 0.05 mM, 0.1 mM, 0.2 mM or 0.4 mM with respect to the initial inoculation amount (O.D.=0.1) of the strains in the whole culture mediums. The concentration of MV might be below 4.0 mM, 3.0 mM, 2.0 mM, 1.0 mM or 0.6 mM in the whole culture mediums. For example, the concentration of MV might be in a range of 0.01 to 4.0 mM in the whole culture mediums. The concentration of MV might be in a range of 0.05 to 3.0 mM in the whole culture mediums. The concentration of MV might be in a range of 0.1 to 2.0 mM in the whole culture mediums. The concentration of MV might be in a range of 0.2 to 1.0 mM in the whole culture mediums. The concentration of MV might be in a range of 0.4 to 0.6 mM in the whole culture mediums. If the concentration of MV is too low, the effects become bad, and if the concentration of MV is too high, the growth of the microorganism might be slow.

The method for enhancing the production of butyrate comprises a step of enhancing the production of butyrate in such a way to add electron transfer mediators to the culture medium of Clostridium tyrobutyricum and to ferment by providing electrons to the culture medium using a reducing electrode(cathode).

The method for enhancing the production of butyrate comprises a step of determining a reduction potential which is supplied to the reduction electrode depending on the electron transfer mediators which will be used with a cyclic voltammogram and to supply the determined reduction potential. The optimum reduction potential can change depending on the environment conditions. It is possible to more enhance the production of butyrate in such a way to previously determine the optimum reduction potential under a corresponding condition and to supply the determined reduction potential.

The electron transfer mediator might be a Neutral Red (NR).

It is not expected to achieve an enhancement of the production of butyrate in such a simple way to add NR whereas the reduction electrode system had an increase in the production of butyrate. In contrast, the MV simply added to the culture medium has appeared to enhance the production of butyrate; however the MV can be reduced thanks to the reduction electrode in the reduction electrode system while not contributing to the enhancement in the production of butyrate of C. tyrobutyricum. In other words, the electrons are not actually transferred to the microorganisms.

The method for enhancing the production of butyrate includes a step of cultivating Clostridium tyrobutyricum strains in the electrode system formed of an oxidation electrode (anode) and a reduction electrode (cathode). The reduction electrode might contain a culture medium having an added electron transfer mediator such as a Neutral Red (NR), etc.

The above mentioned electrode system might further include a positive ion exchange membrane. Namely, the oxidation electrode and the reduction electrode might be separated by the positive ion exchange membrane.

The reduction electrode might be a chamber under an anaerobic environment.

The addition amount of the NR might be above 0.01 mM, 0.05 mM or 0.08 mM with respect to the initial inoculation amount (O.D.=0.1). In addition, the concentration of the NR might be below 1.0 mM, 0.5 mM or 0.2 mM. For example, the concentration of the NR might be in a range of 0.01 to 1.0 mM in the whole culture medium. In addition, the concentration of the NR might be in a range of 0.05 to 0.5 mM in the whole culture medium. In addition, the concentration of the NR might be in a range of 0.08 to 0.2 mM in the whole culture medium. If the concentration of the NR is too low, the effects become bad, and if the concentration of the NR is too high, the growth of the microorganism might be inhibited. The optimum concentration of the NR is 0.1 mM when the initial inoculation amount (O.D.) is 0.1.

The electrode system might be a three-electrode system formed of an oxidation electrode, a reduction electrode and a reference electrode. The oxidation electrode might be a platinum electrode, but it is not limited thereto. The above mentioned reference electrode might be Ag/AgCl electrode, but it is not limited thereto.

In the above mentioned method, the electrode system can adjust pH during the production of butyrate. The proper pH is in a range of 5 to 7 or 5.5 to 6.5 or 5.7 to 6.3. The optimum pH is about 6.

In the above mentioned method, the electrode system might not adjust pH during the production of butyrate. Even though pH is not adjusted, the production of butyrate can be enhanced to a certain level.

The present invention will be described in more details along with the embodiments of the present inventions; however it should be understood that such descriptions are provided for illustrative purposes while not limiting the scopes of the invention.

### 1. Separation of Butyrate Production Strains

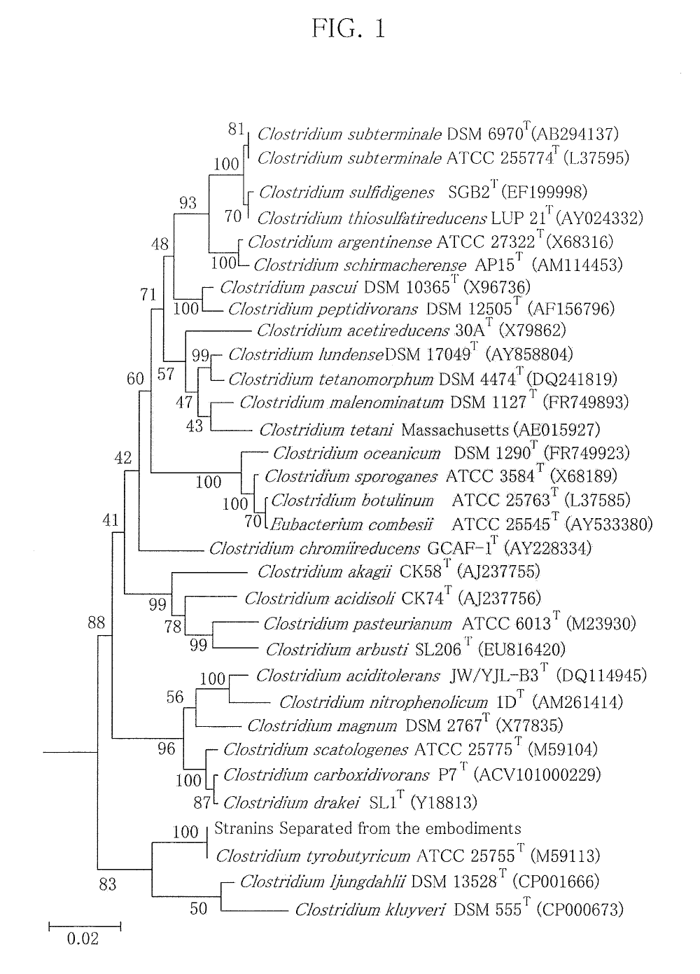

1 g of anaerobic sludgy was taken at Chungrang Water Recycling Center located at Sungdong-gu, Seoul, Korea, and the collected sludgy was heat-treated at 90° C. for 30 minutes, by which a gram negative bacillus was eliminated. The heat-treated anaerobic sludgy was inoculated on a MP2 liquid medium which is a limiting medium (sucrose 20 g, K₂HPO₄ 0.5 g, KH₂PO₄ 0.5 g, Yeast extract 1 g, Ammonium acetate 2.2 g, MgSO₄ 0.2 g, MnSO₄ 0.01 g, I-cysteine HCl 0.5 g) and was enrichment-cultivated at 37° C. for 3 days. The enrichment-cultivated culture liquid was smeared on the MP2 culture medium containing 20 g of sodium butyrate, and colonies resistant to butyrate were selected. Each selected clone was inoculated on the MP2 liquid culture medium and was cultivated for 3 days at 37° C., and the production amount of butyrate was measured by using a Gas Chromatography (GC). As a result of the measurement, the microorganism which produced the maximum amount was separated. As a result of the base sequence analysis of 16S rRNA gene of the strain in the separated clostridium, it was confirmed that it belonged to the Clostridium tyrobutyricum as shown in the dendrogram of FIG. 1.

### 2. Experimental Example 1

### Effects of Enhancement of Production of Butyrate by the Addition of Electron Transfer Medium

### Embodiment 1-1 to Embodiment 1-7

The P2 culture medium (Qureshi and Blaschek 1999) of pH 6.4±0.1 containing sucrose of 20 g/L instead glucose was sterilized. The Methyl Viologen (MV) (E°′=−446 mV) (Aldrich, South Korea) was added to the sterilized P2 culture medium with the concentrations of 0.1 mM (embodiment 1-1), 0.4 mM (embodiment 1-2), 0.5 mM (embodiment 1-3), 1.0 mM (embodiment 1-4), 2.0 mM (embodiment 1-5), 5.0 mM (embodiment 1-6) and 10.0 mM (embodiment 1-7). The separated Clostridium tyrobutyricum strains were inoculated on the MV-added P2 culture medium and were cultured at 37° C. and 150 rpm.

### Comparison Example 1

The strains were cultured under the same conditions as the conditions of the embodiments 1-1 to 1-7 except for the strains which didn't have an added MV.

### Comparison Examples 2-1 to 2-2

The strains were cultured under the same conditions as the conditions of the embodiments 1-1 to 1-7 except for the strains on which 0.1 mM and 0.5 mM of the Neutral Red (NR) (E°′=−325 mV) (Aldrich, South Korea) were added instead of the MV.

### Comparison Examples 3-1 to 3-7

The strains were cultured under the same conditions as the conditions of the embodiments 1-1 to 1-7 except for the strains on which 0.5 mM of anthraquinone-1,5-disulfonic acid, AQDS) (E°′=−184 mV) (Aldrich, South Korea) was added instead of the MV.

The butyrate and acetate produced through the embodiments 1-1 to 1-7, the comparison example 1, the comparison examples 2-1 to 2-7 and the comparison examples 3-1 to 3-7 were analyzed by HP-INNOWax column (30 m×250 μm×0.25 μm, Agilent Technologies and the gas chromatography (GC, Agilent technology 6890N Network GC System) with the frame-ionized detector. The hydrogen and carbon dioxide were quantified by multiplying the gas composition with the increased gas volume. The gas composition percentage was analyzed by the GC having a heat conductivity detector. The consumed sucrose concentration was quantified by a high performance liquid chromatography (HPLC) (Hi-plex H, Agilent, Santa Clara, Calif.) with 5 mM of sulfuric acid as a mobile phase at the flow rate of 0.5 mLmin⁻¹. The growth of microorganisms was checked at 600 nm of absorbance by using Shimadzu UVmini-1240 spectrophotometer or was checked with the Chemical Oxygen Demand (COD, mg/L unit) induced by the base soluble COD from the total COD by using COD vials (C-mac, South Korea, concentration range of 10-1,500 mg/L). The obtained values were average values obtained as the experiments were repeatedly performed three times or four times under a corresponding condition.

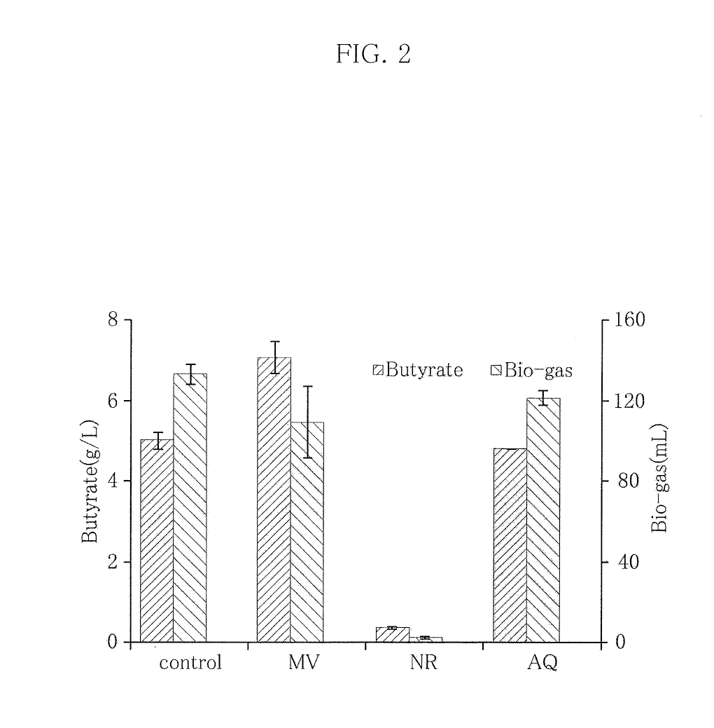

Table 1 and FIG. 2 show a result of the experiments.

TABLE 1

| | | Butyrate | | | | Bio-gas | | | |
|---|---|---|---|---|---|---|---|---|---|
| Methyl | OD | final | | % vs. | | | | % vs. | |
| viologen (mM) | (600 nm) | conc. (g/L) | | control | | vol (mL) | | control | |
| 0 (comparison example 1) | 5.9 (±0.4) | 5.7 | (±0.6) | 100 | | 134 | (±8) | 100 | |
| 0.1(embodiment 1-1) | 6.2 (±0.4) | 6.5 | (±0.5) | 115 | (±9) | 110 | (±9) | 82 | (±7) |
| 0.4(embodiment 1-2) | 6.3 (±0) | 7.3 | (±0.1) | 128 | (±2) | 107 | (±11) | 80 | (±8) |
| 0.5(embodiment 1-3) | 5.8 (±0.3) | 8.5 | (±0.4) | 150 | (±7) | 87 | (±4) | 65 | (±3) |
| 1(embodiment 1-4) | 5.4 (±0.8) | 6.2 | (±1.7) | 109 | (±30) | 67 | (±1) | 50 | (±1) |
| 2(embodiment 1-5) | 3.9 (±0.9) | 6.4 | (±1.2) | 112 | (±22) | 46 | (±0) | 34 | (±9) |
| 5(embodiment 1-6) | 1.5 (±1.9) | 0.8 | (±0) | 14 | (±0) | 4 | (±0) | 3 | (±0) |
| 10(embodiment 1-7) | 0.2 (±0.1) | 0.7 | (±0) | 12 | (±0) | 4 | (±0) | 3 | (±0) |

As seen in Table 1, it is known that when the MV has a proper concentration, the production of butyrate is increased.

FIG. 2 is a graph showing the results of the embodiment 1-3(MV), the comparison example 2-3(NR) and the comparison example 3-3(AQDS) after MV, NR and AQDS were added on the culture mediums with the concentration of 0.5 mM. As shown in FIG. 2, when the MV was used as the electron transfer mediator, the production of butyrate was highest. Most of the strains need an electron transfer mediator so as to receive electrons from the reduction electrode. The above-mentioned results represent that the production of butyrate can be enhanced by simply adding the electron transfer mediator without using the reduction electrode. In other words, when the MV was used as the electron transfer mediator, it was possible to enhance about 1.5 times the final concentration of butyrate as compared to the comparison group in such a way that the surplus electrons were involved in the production of butyrate by inhibiting the production of hydrogen by way of a simple addition.

### 3. Experimental Example 2

### Enhancement of Production of Butyrate with Reduction Electrode

### Embodiment 2

A Bioelectrical Reactor (BER) formed of Pyrex was manufactured, which was specially designed for the sake of the anaerobic bacteria culture by changing a little the H-type reactor with a dual chamber (450 mL each). The reduction electrode (cathode) was a graphite felt electrode (4.5 cm×12 cm), and the oxidation electrode (anode) was a Pt plate electrode (2.5 cm×5 cm). The reference electrode was Ag/AgCl, 3 M KCl (BASi, West Lafayette, Ind.) and was dipped at a portion of the reduction electrode. The portions of the oxidation electrode and the reduction electrode were separated by Nafion 117 positive ion-exchange membrane (Naracell-tech, South Korea). The biological gas was collected by a Tedlar bag connected with a stainless steel fitting inserted in a reduction electrode stopper. The temperature of the reactor was maintained at 37±1° C. by a heating tape (Daihan, South Korea). The temperature sensor was inserted into a partition of the oxidation electrode by way of the stainless steel stand rod.

The portion of the reduction electrode of the reactor has features in that the neutral red was filled with the P2 medium added with 0.1 mM, and the portion of the oxidation electrode was filled with hexacyanoferrate [K₄(Fe(CN)₆)·3H₂O]. The BER operated under a constant reduction electrode potential of −400 mV (vs. Ag/AgCl). The above mentioned potential was set by the potentiostat/galvanostat (EG&G Princeton Applied Research, Model 273A, Princeton, N.J.) which could be controlled by a computer. So, the current between the oxidation electrode and the reduction electrode was monitored. The reduction voltage applied to the reduction electrode was determined from the redox peak in the cyclic voltammogram of the culture liquid to which the neutral red was added at the scan speed of 20 mV/s based on the reduction electrode versus Ag/AgCl. The reduction of the neutral red by the reduction electrode appeared as the color of the solution turned from the red (NRₒₓ) to the yellow (NR_{red}) when it was balanced to the negative potential within 5-30 minutes. For the sake of pH-adjusted BER operation, pH was maintained at 6 by temporarily adding 3M NaOH.

### Embodiment 3

The embodiment 3 was performed in the same manner as the embodiment 2 except for the adjustment of pH.

### Embodiment 4

The embodiment 4 was performed in the same manner as the embodiment 2 except that the portion of the reduction electrode of the reactor was filed with the P2 medium added with the MV of 0.5 mM of concentration was operated under a constant reduction electrode potential of −750 mV (vs. Ag/AgCl). In case of the MV, the reduction had features in that the color of the solution turned from a colorlessness (MVₒₓ) to a purple color (MV_{red}).

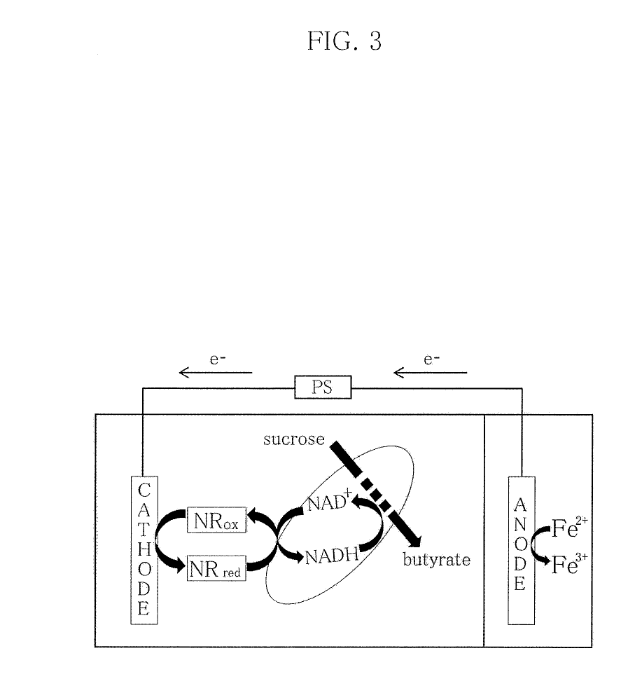

The results of the comparison example 1 and the embodiments 2 to 4 are shown in Table 2 and FIGS. 3 to 5.

TABLE 2

| | | | Embodi- | |
|---|---|---|---|---|
| | | Embodi- | ment 3 | |
| | Compari- | ment 2 | BER | Embodi- |
| | son ex- | (BER⁴) | (no pH | ment 4 |
| | ample 1 | (pH 6⁵) | control) | (MV³) |
| Final pH | 4.3 ± 0.1 | 6.0 ± 0.1 | 4.3 ± 0.1 | 4.3 ± 0.1 |
| Specific growth | 0.10 ± 0.01 | 0.87 ± 0.64 | 0.11 ± 0.01 | 0.10 ± 0.01 |
| rate (h⁻¹) | | | | |
| Sucrose uptake | 0.94 ± 0 | 0.67 ± 0 | 0.64 ± 0.31 | 0.85 ± 0.05 |
| rate¹ (g/L, h⁻¹) | | | | |
| Butyrate | 0.27 ± 0 | 0.28 ± 0.01 | 0.31 ± 0.07 | 0.34 ± 0.01 |
| productivity² | | | | |
| (g/L, h⁻¹) | | | | |
| Acetate | 0.44 ± 0 | 0 | 0 | 0.25 ± 0 |
| productivity² | | | | |
| (g/L, h⁻¹) | | | | |
| Maximum | 6.4 ± 0.3 | 5.1 ± 0.7 | 5.0 ± 0.4 | 5.3 ± 0.4 |
| O.D. (600 nm) | | | | |
| Final butyrate | 5.0 ± 0.2 | 8.8 ± 0.9 | 6.7 ± 0.3 | 7.1 ± 0.4 |
| concentration | | | | |
| (g/L) | | | | |
| Butyrate yield | 0.33 ± 0.02 | 0.44 ± 0.04 | 0.45 ± 0.02 | 0.43 ± 0.02 |
| (g butyrate/g | | | | |
| sucrose) | | | | |

¹maximum uptake rate
²maximum productivity
³MV; methyl viologen (0.5 mM)
⁴BER; bioelectrical reactor with neutral red
⁵pH-controlled BER

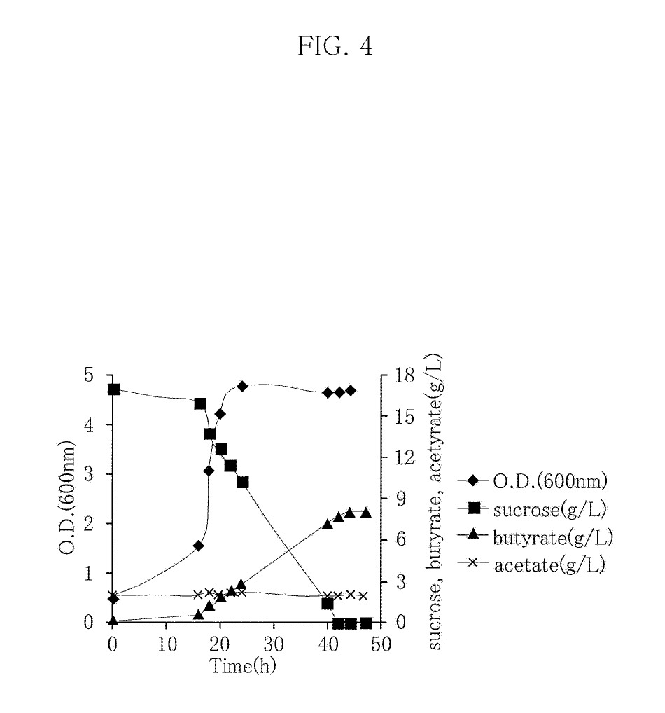

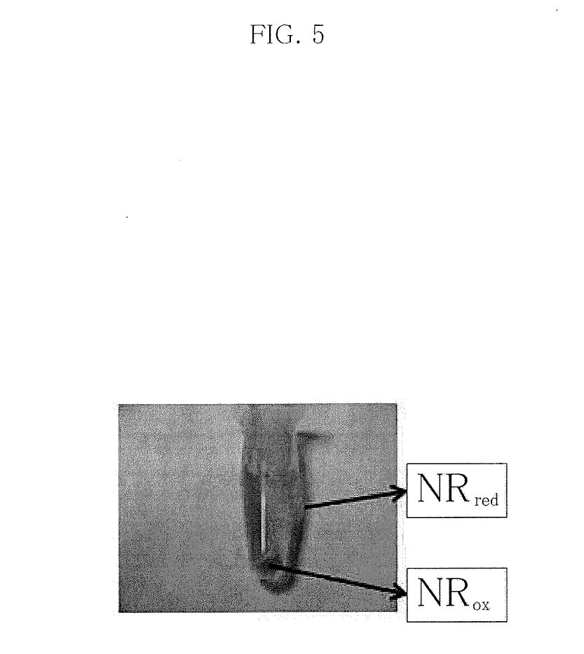

As seen in Table 2, the reduction electrode system (embodiments 2 and 3) did not produce acetic acid, and butyrate was only produced as a liquid fermentation substance, which meant that more electrons were supplied thanks to the production of butyrate. The MV simply inputted in the medium helped enhance the production of butyrate (refer to the embodiment 1 of the experimental example 1), whereas the reduction electrode system (embodiment 4) had features in that the MV was reduced by the reduction electrode (−750 mV vs. Ag/AgCl) (in other words, it was reduced and turned purple); however it didn't help enhance the production of butyrate of C. tyrobutyricum. More specifically speaking, the electron transfers to the microorganism were failed. In contrast, the neutral red (comparison example 2 of experimental example 1), which didn't make any effects when it was simply inputted in the medium, showed an increase in the production of butyrate in terms of the reduction electrode system (embodiments 2 and 3). FIG. 3 shows the reduction electrode system by the neutral red. A result of the embodiment 2 is shown in the graph of FIG. 4. Finally, the production of butyrate was increased from 5 g/L to 8 g/L (FIG. 4). As shown in FIG. 5, it was reduced from the red oxidation type of NRₒₓ to the yellow reduction type of NR_{red} by way of the reduction electrode (−400 mV vs. Ag/AgCl) (refer to FIG. 5).

What is claimed is:

1. A method for enhancing a butyrate production by Clostridium tyrobutyricum strains, comprising:
adding an electron transfer mediator on a medium of Clostridium tyrobutyricum strains and fermenting the same; or
providing electrons to the Clostridium tyrobutyricum strains with the aid of a reduction electrode (cathode) and fermenting the same for thereby enhancing the production of butyrate.

2. The method of claim 1, wherein the method for enhancing a butyrate production by Clostridium tyrobutyricum strains is directed to enhancing the production of butyrate while reducing the production of byproducts or not producing byproducts.

3. The method of claim 2, wherein the byproduct is an acetic acid.

4. The method of claim 1, wherein the electron transfer mediator is a Methyl Viologen (MV).

5. The method of claim 4, wherein the concentration of the methyl viologen is in a range of 0.01 to 4.0 mM of the total mediums when the initial inoculation amount (O.D.) of strains is 0.1.

6. The method of claim 5, wherein the concentration of the methyl viologen is in a range of 0.1 to 2.0 mM of the total mediums when the initial inoculation amount (O.D.) of strains is 0.1.

7. The method of claim 1, wherein the method for enhancing a butyrate production by Clostridium tyrobutyricum strains comprises:
adding an electron transfer mediator to a medium of Clostridium tyrobutyricum strains; and
providing electrons to the medium with the aid of a reduction electrode (cathode) and fermenting the same for thereby enhancing the production of butyrate.

8. The method of claim 7, wherein the method for enhancing a butyrate production by Clostridium tyrobutyricum strains comprises:
determining a reduction potential supplied to the reduction electrode in accordance with an electron transfer mediator to be adapted by using a cyclic voltammogram; and
supplying the determined reduction potential.

9. The method of claim 7, wherein the method for enhancing a butyrate production by Clostridium tyrobutyricum strains comprises:
cultivating Clostridium tyrobutyricum strains in an electrode system formed of an oxidation electrode (anode) and a reduction electrode (cathode), the reduction electrode including a medium on which an electron transfer mediator is added.

10. The method of claim 9, wherein the electrode system further comprises a positive ion exchange membrane, the oxidation electrode and the reduction electrode being separated by a positive ion exchange membrane.

11. The method of claim 7, wherein the electron transfer mediator is a Neutral Red (NR).

12. The method of claim 7, wherein the method for enhancing a butyrate production by Clostridium tyrobutyricum strains comprises:
adjusting pH in the electrode system during the production of butyrate.

13. The method of claim 7, wherein the method for enhancing a butyrate production by Clostridium tyrobutyricum strains comprises:
not adjusting pH in the electrode system during the production of butyrate.
